(19) United States
(12) Patent Application Publication
(10) Pub. No.: US 2006/0287589 A1
(43) Pub. Date: Dec. 21, 2006

(54) DIGITAL PHOTOPLETHYSMOGRAPHIC SIGNAL SENSOR

(76) Inventors: James Wobermin, Arvada, CO (US); Mark A. Norris, Louisville, CO (US); D. Alan Hanna, Boulder, CO (US)

Correspondence Address: MARSH, FISCHMANN & BREYFOGLE LLP, 3151 SOUTH VAUGHN WAY, SUITE 411, AURORA, CO, 80014, US

(21) Appl. No.: 11/423,091

(22) Filed: Jun. 8, 2006

Related U.S. Application Data
(60) Provisional application No. 60/691,051, filed on Jun. 16, 2005.

(51) Int. Cl. A61B 5/00

(52) U.S. Cl. 600/324

(57) ABSTRACT

A photoplethysmographic sensor and related method for use with a photoplethysmographic instrument such as a pulse oximeter are provided. In accordance with the present invention, the detector output signal from the sensor is digitized prior to communication from the sensor to the instrument and the sensor operates independent of the instrument with respect to controlling the light signal emitters of the sensor. In one embodiment, the digitized detector output signal is communicated to the instrument via a wireless communication link.

### CROSS REFERENCE TO RELATED APPLICATION

This application claims priority under 35 U.S.C. §119 to U.S. Provisional Application No. 60/691,051 entitled “Digital Photoplethysmographic Signal Sensor” having a filing date of Jun. 16, 2005, the entire contents of which are incorporated herein by reference.

### FIELD OF THE INVENTION

The present invention relates generally to photoplethysmography, and more particularly to a sensor for use with photoplethysmographic instruments that outputs a digital signal to the instrument.

### BACKGROUND OF THE INVENTION

Signal attenuation measurements generally involve transmitting a signal towards or through a medium under analysis, detecting the signal transmitted through or reflected by the medium and computing a parameter value for the medium based on attenuation of the signal by the medium. In simultaneous signal attenuation measurement systems, multiple signals are simultaneously transmitted (e.g., two or more signals are transmitted during at least one measurement interval) to the medium and detected in order to obtain information regarding the medium.

Such attenuation measurement systems are used in various applications in various industries. For example, in the medical or health care field, optical (e.g., visible spectrum or other wavelength) signals are utilized to monitor the composition of respiratory and anesthetic gases, and to analyze a tissue or a blood sample with regard to oxygen, carbon dioxide or other gas saturation levels, analyte values (e.g., related to certain hemoglobins) or other composition related values. Signal attenuation measurement systems using optical or light signals are often referred to as photoplethysmographic instruments, and one example of a photoplethysmographic instrument is a pulse oximeter.

Pulse oximeters determine the levels of oxygen and/or other gases in a patient's blood, or related analyte values, based on transmission/absorption characteristics of light transmitted through or reflected from the patient's tissue. Pulse oximeters also determine the patient's pulse rate from information included in one or more of the attenuated light signals. In particular, pulse oximeters generally include a probe or sensor for attaching to a patient tissue site such as, for example, a finger, earlobe, nasal septum, or foot. The probe is used to transmit pulsed light signals of at least two wavelengths, typically red and infrared, to the patient tissue site. The light signals are attenuated by the patient tissue site. The attenuated light signals are also often referred to as the transmitted signals, and the transmitted signals are received by a detector that provides an analog electrical output signal representative of the received optical signals. By processing the electrical signal output by the detector and analyzing signal values for each of the wavelengths at different portions of a patient pulse cycle, information can be obtained regarding blood gas saturation levels. As may be appreciated, a multiplexing technique (such as time division, frequency division, code division, or a combination of these techniques) may be employed to drive the light signal emitters in order facilitate obtaining information relating to each of the transmitted light signals from the detector output signal.

Typical sensors include the light signal emitters and the detector in conjunction with a positioner and a cable for connecting the sensor to the photoplethysmographic instrument. As may be appreciated, the cable typically includes a number of conductors for transmitting drive signals from the instrument to the light signal emitters to control their operation in accordance with the employed multiplexing technique, a conductor for communicating the analog detector output signal to the instrument for further processing thereby, and a common conductor. The cable may also have one or more sense wires for use in monitoring the operations of the light signal emitters (e.g., measuring their resistance). The various signals transmitted via the conductors in the cable, and in particular the analog detector output signal, are susceptible to electromagnetic signal interference from various sources, including other electrically powered equipment often present in hospital rooms and other facilities where patients are treated. Furthermore, the relatively narrow gauge conductors in the probe cables can sometimes be fragile resulting in defective probes.

### SUMMARY OF THE INVENTION

Accordingly, the present invention is directed to a sensor and related method for use with a photoplethysmographic instrument such as a pulse oximeter wherein the detector output signal is digitized prior to communication from the sensor to the instrument. Additionally, the present invention is directed to a sensor and related method for use with a photoplethysmographic instrument such as a pulse oximeter wherein the sensor operates independent of the instrument with respect to controlling the light signal emitters or the like in generating and multiplexing the necessary light signals. Further, the present invention is also directed to a sensor and related method for use with a photoplethysmographic instrument such as a pulse oximeter wherein the digitized detector output signal is communicated to the instrument via a wireless communication link.

The present invention achieves a number of advantages. By digitizing the detector output signal, the potential for corruption of the detector output signal during transmission from the sensor to the monitor due to electromagnetic signal interference or the like is reduced. By controlling operation of the light signal emitters onboard the sensor, at least two conductors can be eliminated in embodiments with a cable connecting the sensor to the instrument, and a wireless connection between the sensor and instrument is permitted. By employing a wireless communication link between the sensor and the instrument to communicate the digitized detector output signal, greater patient mobility is allowed and the instrument may be located at greater distance from the patient.

The aforementioned features and advantages of the present invention are achieved by a number of aspects of the present invention. According to one aspect of the present invention a photoplethysmographic sensor for use with a photoplethysmographic instrument such as, for example, a pulse oximeter, includes at least first and second light signal emitters, a detector and a signal processing device. The light signal emitters, detector, and signal processing device (and other components of the sensor) may all be incorporated into a positioner configured for attachment to a patient tissue site that positions the first and second light signal emitters and the detector in an appropriate relation with one another and the patient tissue site.

The first and second light signal emitters are operable to transmit at least first and second light signals centered at first and second wavelengths (e.g., Red and Infrared), respectively, into a tissue site of a patient. The patient tissue site attenuates the first and second light signals resulting in first and second attenuated light signals. The detector is operable to detect the first and second attenuated light signals and to output an analog detector output signal corresponding to the first and second attenuated signals. The signal processing device is operable to receive the analog signal from the detector and to generate a digital signal corresponding to the analog detector output signal. The digital signal is communicable to the photoplethysmographic instrument whereby the photoplethysmographic instrument may obtain information from the digital signal relating to a physiological condition of the patient (e.g., the patient's blood oxygen level and/or pulse rate).

The signal processing device may comprise an electronic device such as a field programmable gate array (FPGA) or an application specific integrated circuit (ASIC), with the FPGA or ASIC configured to incorporate an amplifier and an analog-to-digital converter. The sensor may also include a light signal emitter drive unit operable to control the emission of light signals from the light signal emitters. In this regard, the light signal emitter drive unit may be an FPGA or an ASIC separate from the signal processing device or it may be incorporated as part of an FPGA or ASIC comprising the signal processing device.

The sensor may be configured to communicate the digital signal to the photoplethysmographic instrument via a wired communication link. In this regard, the sensor may include a cable connectable with an input of the photoplethysmographic instrument. Where the photoplethysmographic instrument is configured to receive an analog input signal, the sensor may be accompanied by an adaptor unit connectable with the input of the instrument and the cable of the sensor that converts the digital signal from the sensor to an analog signal.

The sensor may also be configured to communicate the digital signal to the photoplethysmographic instrument via a wireless communication link. In this regard, the sensor may also include a wireless transmitter operable to communicate the digital signal to the photoplethysmographic instrument via the wireless communication link (e.g., radio-frequency or free-space optical). In order to accommodate wireless communication of the digital signal, the photoplethysmographic instrument needs to be configured to receive the digital signal via the wireless communication link by, for example, including a wireless receiver within the instrument. Alternatively, the sensor may be accompanied by a separate wireless receiver unit that is configured to connect to an input of the photoplethysmographic instrument. The wireless receiver unit adapts the photoplethysmographic instrument to receive the digital signal via the wireless communication link. Where a wireless communication link is employed between the sensor and the instrument, it may be desirable to encode the digital signal prior to communication of the digital signal via the wireless communication link in order to facilitate association of the digital signal with the particular sensor, particularly in environments where other digital photoplethysmographic sensors may be present. Further, the sensor may include a power source such as, for example, a battery, in order to provide electrical power to the various electronic components of the sensor.

According to another aspect of the present invention, a system for obtaining information relating to a physiological condition (e.g., blood oxygen level and/or pulse rate) of a patient based on information derived from light signals attenuated by a tissue site of the patient includes a sensor and a monitor. The sensor is operable to generate and direct at least two light signals at the patient tissue site, with the light signals being centered at different wavelengths (e.g., Red and Infrared). The sensor is also operable to detect the light signals after being attenuated by the patient tissue site and to digitize the detected attenuated light signals. The monitor includes a digital signal processor that is operable to receive the digitized detected attenuated light signals and to process the digitized detected attenuated light signals to obtain the patient physiological condition therefrom.

The sensor may include at least two light signal emitters operable to emit the light signals, a light signal emitter drive unit coupled to the light signal emitters and operable to control the emission of light signals from the light signal emitters, a detector operable to the detect attenuated light signals and to output an analog detector output signal corresponding to the attenuated signals, and an analog-to-digital converter coupled to the detector and operable to digitize the analog detector signal. The sensor may also include an amplifier coupled between the detector and the analog-to-digital converter. The analog-to-digital converter and the amplifier may be implemented within a first electronic component (e.g., an FPGA or ASIC), and the light signal emitter drive unit may be implemented within a second electronic component (e.g. another FPGA or ASIC). The light signal emitter drive unit, analog-to-digital converter, and amplifier may instead all be implemented within a single electronic component (e.g., FPGA or ASIC).

The sensor may also include a wireless transmitter operable to communicate the digitized detected attenuated light signals to the photoplethysmographic instrument via a wireless communication link (e.g., radio-frequency or free-space optical). In this regard, the monitor may include a wireless receiver operable to receive the digitized detected attenuated light signals via the wireless communication link or the system may further include a wireless receiver unit configured to connect to an input of the monitor to adapt the monitor to receive the digitized detected attenuated light signals via the wireless communication link. The sensor may also include a cable configured to communicate the digitized detected attenuated light signals to the monitor. In this regard, the system may further include an adaptor unit connectable with an input of the monitor and with the cable of the sensor that is operable to convert the digitized detected attenuated light signals receivable from the cable of the sensor to an analog signal transmittable from the adaptor unit to the input of the monitor.

According to yet another aspect of the present invention, a method for use in obtaining information relating to a physiological condition of a patient from light signals attenuated by a tissue site of the patient includes the step of operating a sensor located at a patient tissue site to direct at least two light signals (e.g., Red and Infrared light signals) into the patient tissue site. Operating the sensor also involves detecting the light signals after the light signals are attenuated by the patient tissue site and generating a digital signal corresponding to the attenuated light signals. In accordance with the method, the digital signal is communicated to a monitor separate from the sensor. In this regard, the digital signal may be communicated using a wired communication link or a wireless communication link between the sensor and the monitor. The method may also include adapting the monitor to receive the digital signal via the wireless communication link or adapting the monitor to receive the digital signal via the wired communication link. However received, the digital signal is processed at the monitor to obtain information relating to the patient physiological condition (e.g., blood oxygen level and/or pulse rate).

These and other aspects and advantages of the present invention will be apparent upon review of the following Detailed Description when taken in conjunction with the accompanying figures.

### DESCRIPTION OF THE DRAWINGS

For a more complete understanding of the present invention and further advantages thereof, reference is now made to the following Detailed Description, taken in conjunction with the drawings, in which:

FIG. 1 is a block diagram of a pulse oximetry system incorporating one embodiment of a digital photoplethysmographic sensor in accordance present invention;

FIG. 2 is a block diagram showing the digital photoplethysmographic sensor of FIG. 1 in greater detail;

FIG. 3 is a block diagram of another pulse oximetry system incorporating a wireless embodiment of a digital photoplethysmographic sensor in accordance present invention;

FIG. 4 is a block diagram showing the digital photoplethysmographic sensor of FIG. 3 in greater detail;

FIG. 5 is a block diagram of another pulse oximetry system incorporating a wireless embodiment of a digital photoplethysmographic sensor and having a wireless receiver adaptor unit in accordance present invention; and

FIG. 6 is a block diagram of another pulse oximetry system incorporating a wired embodiment of a digital photoplethysmographic sensor and having a digital-to-analog adaptor unit in accordance present invention.

### DETAILED DESCRIPTION

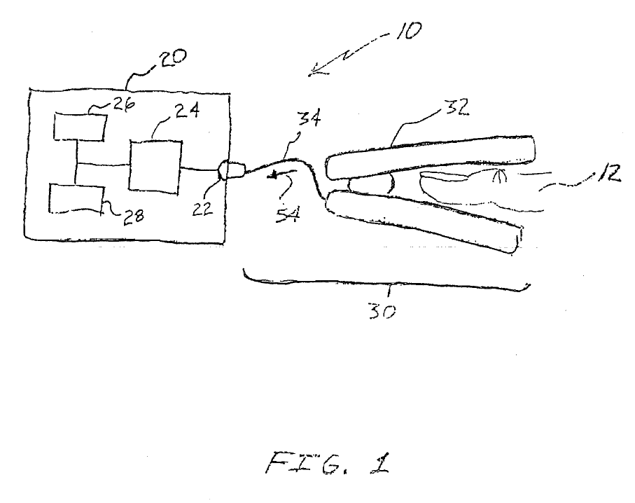

Referring to FIG. 1, one embodiment of a pulse oximetry system 10 incorporating a digital photoplethysmographic sensor 30 is shown. The pulse oximetry system 10 includes a pulse oximeter monitor 20 including an input connector 22, a processor 24, a display 26, and a printer 28. The sensor 30 includes a positioner 32 and a cable 34 shown connected with the input 22 of the monitor 20. The positioner 32 is configured for attachment to a patient tissue site 12. In this regard, the positioner 32 may, for example, be a clip-type positioner such as shown, although other configurations may be utilized as well. The input 22 of the monitor 20 receives a digital signal 54 from the positioner 32 via cable 34. The digital signal 54 is processed by the processor 24 of the monitor 20 to obtain information regarding physiological conditions of the patient such as the patient's blood gas saturation levels as well as the patient's pulse rate. Such physiological conditions may be output on the display 26 and/or printed by the printer 28 on a paper roll or the like.

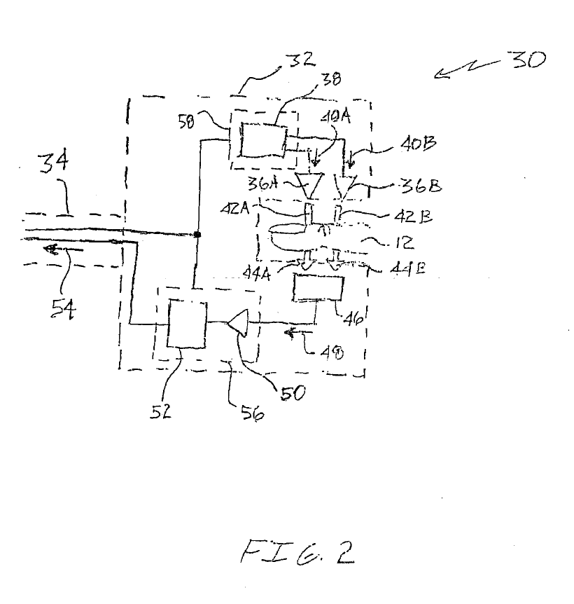

Referring now to FIG. 2, the sensor 30 includes two light signal emitters 36A and 36B, although in other embodiments there may be fewer or more than two light signal emitters. The light signal emitters 36A, 36B may, for example comprise light-emitting diodes (LEDs), laser diodes, or the like. When excited the light signal emitters 36A, 36B emit light centered around different respective first and second wavelengths, such as Red and Infrared, although other wavelength emitters may be employed depending on the intended use of the photoplethysmographic sensor 30. The light signal emitters 36A, 36B are also referred to herein and the Red and Infrared LEDs 36A, 36B.

A light signal emitter drive unit 38 is coupled to the light signal emitters 36A, 36B. The drive unit 38 generates and sends drive signals 40A, 40B to the Red and Infrared LEDs 36A, 36B to cause the LEDs 36A, 36B to emit light signals 42A, 42B in the direction of the patient tissue site 12. In this regard, the drive signals 40A, 40B may be generated in accordance with an appropriate multiplexing scheme in order to multiplex light signals 42A, 42B. The light signals 42A, 42B are, in this embodiment, transmitted through the patient tissue site 12 and attenuated thereby producing attenuated or transmitted light signals 44A, 44B.

The sensor 30 also includes a light signal detector 46 such as, for example, a photodiode or the like. In other embodiments there may be more than one detector, with each detector being tuned to receive only particular light frequencies thereby obviating the need the multiplex the light signals 42A, 42B. The detector 46 receives both transmitted light signals 44A, 44B, and generates an analog composite detector output signal 48. The output signal 48 includes information relating to both of the transmitted light signals 44A, 44B.

The sensor 30 further includes an amplifier 50 and an analog-to-digital (A/D) converter 52. The analog composite detector output signal 48 is directed to the amplifier 50 which amplifies the detector output signal 48. The amplifier 50 may also be configured to filter (e.g., high-pass, low-pass, or bandwidth filter) the detector output signal 48. After amplification/filtering, the detector output signal 48 is directed to the A/D converter 52. The A/D converter 52 converts the amplified/filtered detector output signal 48 to a digital output signal 54. In this regard, the A/D converter should sample the detector output signal 48 at a sufficiently high sample rate (e.g., 30 to 50 Hz) in order to accurately digitize the detector output signal 48 without losing significant information relating to the levels of the transmitted light signals 44A, 44B.

As illustrated, the amplifier 50 and the A/D converter 52 may be implemented within a first signal processing device or electronic component 56, such as, for example, a field programmable gate array (FPGA) or an application specific integrated circuit (ASIC). Likewise, the light signal emitter drive unit 38 may be implemented using a second signal processing device or electronic component 58 such as, for example, another FPGA or ASIC. In other embodiments, the light signal emitter drive unit 38, amplifier 50 and A/D converter 52 may be implemented within a single electronic component such as, for example, an FPGA or an ASIC. Still in other embodiments, other electronic components such as an appropriately programmed general purpose microprocessor might be utilized to implement some or all functionality of the light signal emitter drive unit 38, amplifier 50 and A/D converter 52.

As may be appreciated, various components included in the sensor 30 (e.g., the LEDs 36A, 36B and the FPGAs (or ASICs) 56, 58 comprising the light signal emitter drive unit 38, the amplifier 50 and the A/D converter 52) need electrical power in order to operate. In this regard, the cable 34 may include a conductor for supplying such power from, for example, the monitor unit 20. In addition to a power supply conductor, the cable 34 may also include a conductor for transmitting the digital output signal 54 as well as a common conductor. Since the light signal emitter drive signals 40A, 40B are generated at the sensor 30 by the light signal emitter drive unit 38, conductors for conducting drive signals from the monitor 20 to the sensor 30 are not required.

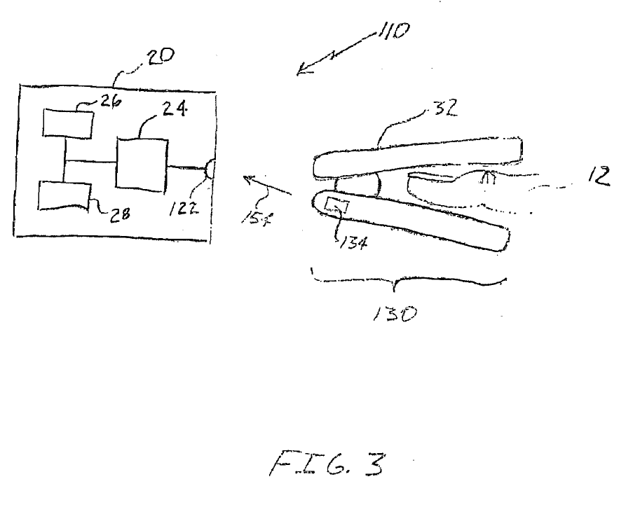

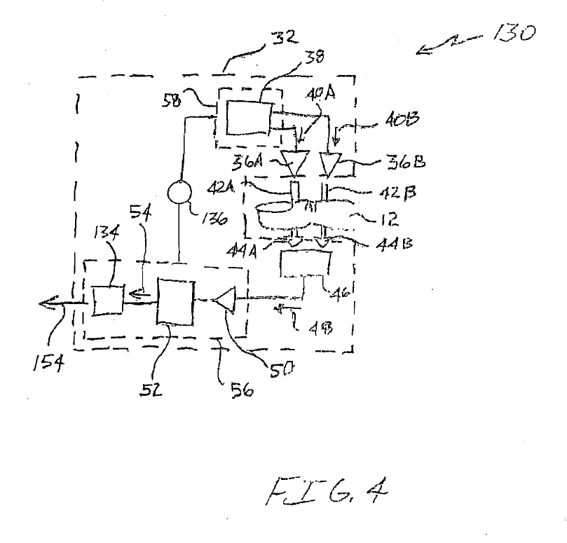

Referring now to FIGS. 3 and 4, another embodiment of a pulse oximetry system 110 incorporating a wireless digital photoplethysmographic sensor 130 is shown. The pulse oximetry system 110 and wireless sensor 130 are configured similar to the pulse oximetry system 10 and sensor 30 illustrated in FIGS. 1 and 2, and similar components are referenced by the same numbers. The pulse oximetry system 110 includes a pulse oximeter monitor unit 20 including a wireless data receiver 122, a processor 24, a display 26, and a printer 28. The sensor 130 includes a positioner 32 and a wireless transmitter 134. The positioner 32 is configured for attachment to a patient tissue site 12, and may, for example, be a clip-type positioner such as shown, although other configurations may be utilized as well. The wireless receiver 122 of the monitor 20 receives a wireless digital signal transmitted by the wireless transmitter 134 from the positioner 32. In this regard, the wireless transmitter 134 and wireless receiver 122 may, for example, comprise radio-frequency (RF) components with the wireless digital signal being an RF signal, or where sufficient line-of-sight conditions can be maintained between the sensor 130 and monitor 20, the wireless transmitter 134 and wireless receiver 122 may, for example, comprise optical components with the wireless digital signal being an optical signal. Regardless of its form, the wireless digital signal received by the wireless receiver 122 is processed by the processor 24 of the monitor 20 to obtain information regarding physiological conditions of the patient such as the patient's blood gas saturation levels as well as the patient's pulse rate. Such physiological conditions may be output on the display 26 and/or printed by the printer 28 on a paper roll or the like.

In addition the various components included in the sensor 30 shown in FIG. 2 (with the exception of a cable), the wireless sensor 130 includes the wireless transmitter 134 and an electrical power source 136 (e.g., a battery) that supplies power for operating the various electronic components of the wireless sensor 130. As is shown, the wireless transmitter may be incorporated within the first electronic component 56 along with the amplifier 50 and A/D converter 52. In other embodiments, the wireless transmitter may be a separate electronic component.

The digital output signal 54 from the A/D converter 52 is directed to the wireless transmitter 134 for transmission to the wireless receiver 122 of the monitor 20. In this regard, the wireless transmitter 134 modulates the digital output signal 54 onto a carrier signal (e.g., RF or optical) to obtain a wireless digital output signal 154 that is transmitted to the wireless receiver 122. The wireless receiver 122 of the monitor 20 receives the wireless digital output signal 154 and demodulates the wireless digital output signal 154 to obtain the digital output signal 54 for further processing by the processor 24 of the monitor 20. By transmitting the digital output signal 54 wirelessly to the monitor 20 without the use of a cable, the patient is permitted greater mobility and monitor 20 does not need to be within a cable's length distance of the patient tissue site 12. In fact, in the case of a RF wireless transmitter 134 and receiver 122, the monitor 20 may not even need to be within the same room as the patient.

Since there may be additional wireless (RF or optical) devices in the same room or area as the patient (e.g., other wireless photoplethysmographic sensors being used with other patients), the sensor 130 may be operable to encode the digital output signal 54 prior to it being modulated onto the carrier signal for transmission. In this regard, the digital output signal 54 may be encoded in a manner that identifies it as being associated with the particular wireless digital photoplethysmographic sensor 130 from which the wireless digital output signal 154 is transmitted. Such functionality may, for example, be incorporated within the first electronic component 56. Upon receipt, the monitor 20 is operable to decode the encoded digital output signal 54. Such functionality may, for example, be included as part of the wireless receiver 122. In order to facilitate decoding, information about the digital photoplethysmographic sensor 130, and in particular the encoding methodology employed, may be provided manually (e.g., by a user) or automatically (e.g., as part of a sensor/monitor initiation sequence) to the monitor 20.

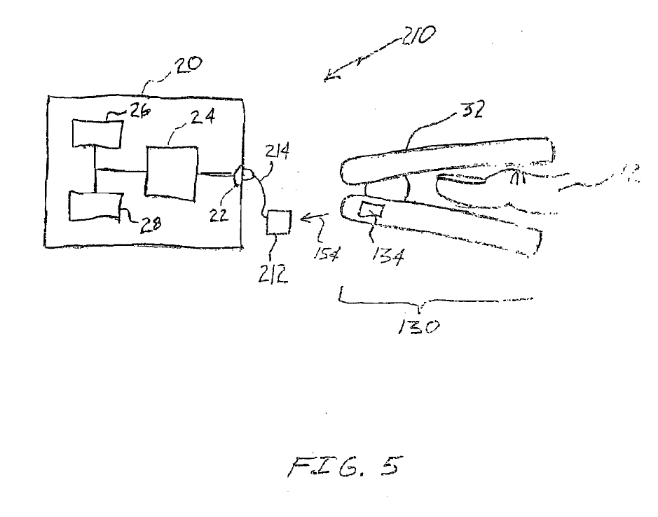

Referring now to FIG. 5, another embodiment of a pulse oximetry system 210 incorporating a wireless digital photoplethysmographic sensor 230 and a wireless adaptor unit 212 is shown. The monitor 20 of the pulse oximetry system 210 is configured similar to the monitor 20 in the pulse oximetry system 10 shown in FIG. 1 and the wireless sensor 130 is configured similar to the wireless sensor 130 of the pulse oximetry system 110 illustrated in FIGS. 3 and 4, and similar components are referenced by the same numbers. The primary difference between the pulse oximetry system 210 shown in FIG. 5 and that shown in FIG. 3 is that the monitor 20 does not include a wireless receiver. Instead, the wireless adaptor unit 212 is connected (via, for example a short cable 214 as shown) with the input connector 22 of the monitor 20. The wireless adaptor unit 212 adapts a monitor 20 which lacks a wireless receiver for receiving the wirelessly transmitted (e.g., RF or optical) digital output signal 154. The adapter unit 212 demodulates the digital output signal 54 from the carrier signal of the wireless digital output signal 154. Where the digital output signal 54 has been encoded, the wireless adaptor unit 212 also may decode the digital output signal 54. The digital output signal 54 is directed to the input 22 of the monitor 20 where after it may be further processed by the processor 24 of the monitor 20. In instances where the monitor 20 is not configured to receive a digital signal, the wireless adaptor unit 212 may also convert the digital output signal 54 to an analog signal for input to the monitor 20 via the input 22 of the monitor 20. This would allow the wireless photoplethysmographic sensor 130 to be utilized with monitors that are configured to receive an analog input signal and include an A/D converter between their input connector and processor.

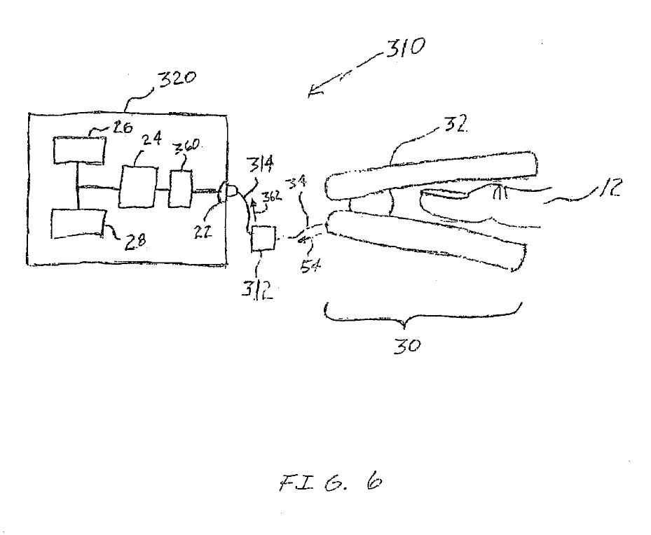

Referring now to FIG. 6, in some instances it may be desirable to utilize a digital photoplethysmographic sensor 30 such as illustrated in FIG. 2 with a pulse oximeter monitor unit configured to receive an analog input signal at the input connector thereof. In this regard, FIG. 6 shows another embodiment of a pulse oximetry system 310 that includes a digital photoplethysmographic sensor 30 having a cable 34 for connecting it to a pulse oximeter monitor unit 320. The monitor unit 320 of the pulse oximetry system 310 shown in FIG. 6 is configured similar to the monitor unit 20 in the pulse oximetry system 10 shown in FIG. 1 and the sensor 30 is configured similar to the sensor 30 illustrated in FIG. 2, and similar components are referenced by the same numbers. One of the differences between the pulse oximetry system 310 shown in FIG. 6 and that shown in FIG. 1 is that the monitor 320 is enabled to receive an analog input signal at the input connector 22 thereof. In this regard, the monitor unit 320 includes an analog-to-digital (A/D) converter 360 between the processor 24 and input connector 22. Further, the pulse oximetry system 310 includes a cabled sensor adaptor unit 312 connected (via, for example a short cable 314 as shown) with the input connector 22 of the monitor 320. The cabled sensor adaptor unit 312 adapts the monitor 320 for receiving the digital output signal 54 from the cable 34 of the sensor 30. The adapter unit 312 converts the digital output signal 54 to an analog signal 362 (e.g., using a digital-to-analog converter included therein) for input to the monitor 320 via the input 22 of the monitor 320. This allows the digital photoplethysmographic sensor 30 to be utilized with monitors that are configured to receive an analog input signal.

In each of the previously described embodiments, since the light signal emitter drive signals 40A, 40B are generated by the sensor (30 or 130), it may be necessary to inform the monitor 20 as to the multiplexing technique being employed so that the processor 24 of the monitor 20 can appropriately demodulate the digital output signal 54 in order to obtain the Red and Infrared transmitted light signals 44A, 44B. One manner of doing so is to add information concerning the multiplexing technique to the digital output signal 54 (e.g., an additional two bits might be added to each word in the digital output signal with the values of the bits providing a code identifying the multiplexing technique utilized). Another possibility is to provide an input informing the monitor 20 of the multiplexing technique (either manually or automatically) to the monitor 20 as part of a monitor/sensor initiation procedure. As an alternative to informing the monitor 20 of the multiplexing technique, separate digital output signals corresponding to each transmitted light signal 44A, 44B may be generated by the sensor 30 or 130 and transmitted to the monitor. In this regard, the sensor 30 or 130 may further include a demodulation unit (not shown) (e.g., as part of the first electronic component 56) that demodulates the digital output signal 54 prior to its transmission to generate separate Red and Infrared digital output signals for transmission to the monitor 20.

While various embodiments of the present invention have been described in detail, further modifications and adaptations of the invention may occur to those skilled in the art. However, it is to be expressly understood that such modifications and adaptations are within the spirit and scope of the present invention.

What is claimed is:

1. A photoplethysmographic sensor for use with a photoplethysmographic instrument, said sensor comprising:
at least first and second light signal emitters operable to transmit at least first and second light signals centered at first and second wavelengths, respectively, into a tissue site of a patient, wherein the tissue site attenuates the first and second light signals resulting in first and second attenuated light signals;
a detector operable to detect the first and second attenuated light signals and to output an analog detector output signal corresponding to the first and second attenuated signals; and
a signal processing device operable to receive the analog signal from said detector and to generate a digital signal corresponding to the analog detector output signal, the digital signal being communicable to the photoplethysmographic instrument whereby the photoplethysmographic instrument may obtain information from the digital signal relating to a physiological condition of the patient.

2. The sensor of claim 1 wherein said processing device includes an analog-to-digital converter and an amplifier.

3. The sensor of claim 2 wherein said signal processing device comprises a field programmable gate array.

4. The sensor of claim 2 wherein said signal processing device comprises an application specific integrated circuit.

5. The sensor of claim 1 further comprising a cable connectable with an input of the photoplethysmographic instrument, the digital signal being communicable from said processor to the photoplethysmographic instrument via said cable.

6. The sensor of claim 5 further comprising:
an adaptor unit connectable with an input of the photoplethysmographic instrument and with the cable of the sensor, said adaptor unit being operable to convert the digital signal receivable from the cable of the sensor to an analog signal transmittable from the adaptor unit to the input of the photoplethysmographic instrument.

7. The sensor of claim 1 further comprising:
a wireless transmitter operable to communicate the digital signal to the photoplethysmographic instrument via a wireless communication link.

8. The sensor of claim 7 wherein the photoplethysmographic instrument is configured to receive the digital signal via the wireless communication link.

9. The sensor of claim 7 further comprising:
a wireless receiver unit configured to connect to an input of the photoplethysmographic instrument and to adapt the photoplethysmographic instrument to receive the digital signal via the wireless communication link.

10. The sensor of claim 7 wherein said wireless transmitter comprises an optical signal transmitter and the wireless communication link comprises an optical link.

11. The sensor of claim 7 wherein said wireless transmitter comprises a radio-frequency signal transmitter and the wireless communication link comprises a radio frequency link.

12. The sensor of claim 7 wherein said signal processing device is further operable to encode the digital signal prior to communication of the digital signal via the wireless communication link.

13. The sensor of claim 7 further comprising:
a power source.

14. The sensor of claim 13 wherein said power source comprises a battery.

15. The sensor of claim 1 further comprising:
a light signal emitter drive unit operable to control the emission of light signals from said light signal emitters.

16. The sensor of claim 15 wherein said light signal emitter drive unit comprises a field programmable gate array.

17. The sensor of claim 15 wherein said light signal emitter drive unit comprises an application specific integrated circuit.

18. The sensor of claim 15 wherein said light signal emitter drive unit and said signal processing device comprise one device.

19. The sensor of claim 1 further comprising:
a positioner configured for attachment to a patient tissue site, the positioner positioning said first and second light signal emitters and said detector in an appropriate relation with one another and the patient tissue site.

20. The sensor of claim 1 wherein the first and second wavelengths are Red and Infrared wavelengths, respectively.

21. The system of claim 1 wherein the patient physiological condition comprises at least one of a blood oxygen saturation level and a pulse rate of the patient.

22. A system operable to obtain information relating to a physiological condition of a patient based on information derived from light signals attenuated by a tissue site of the patient, said system comprising:
a sensor operable to generate and direct at least two light signals at the patient tissue site, the two light signals being centered at different wavelengths, the sensor being further operable to detect the at least two light signals after being attenuated by the patient tissue site and to digitize the detected attenuated light signals; and
a monitor including a digital signal processor operable to receive the digitized detected attenuated light signals and to process the digitized detected attenuated light signals to obtain the at least one patient physiological condition therefrom.

23. The system of claim 22 wherein the at least two light signals are centered at Red and Infrared wavelengths, respectively.

24. The system of claim 22 wherein the patient physiological condition comprises at least one of a blood oxygen saturation level and a pulse rate of the patient.

25. The system of claim 22 wherein said sensor comprises:
at least two light signal emitters operable to emit the light signals;
a light signal emitter drive unit coupled to said light signal emitters and operable to control the emission of light signals from said light signal emitters;
a detector operable to the detect attenuated light signals and to output an analog detector output signal corresponding to the attenuated signals; and
an analog-to-digital converter coupled to said detector and operable to digitize the analog detector signal.

26. The system of claim 25 wherein said sensor further comprises an amplifier coupled between said detector and said analog-to-digital converter.

27. The system of claim 26 wherein said analog-to-digital converter and said amplifier comprise a first electronic component, and wherein said light signal emitter drive unit comprises a second electronic component.

28. The system of claim 27 wherein said first electronic component comprises one of a field programmable gate array and an application specific integrated circuit, and wherein said second electronic component comprises one of a field programmable gate array and an application specific integrated circuit.

29. The system of claim 26 wherein said light signal emitter drive unit, said analog-to-digital converter, and said amplifier comprise a single electronic component.

30. The system of claim 29 wherein said electronic component comprises one of a field programmable gate array and an application specific integrated circuit.

31. The system of claim 22 wherein said sensor includes:
a wireless transmitter operable to communicate the digitized detected attenuated light signals to the monitor via a wireless communication link;
and wherein said monitor includes:
a wireless receiver operable to receive the digitized detected attenuated light signals via the wireless communication link.

32. The system of claim 22 wherein said sensor includes:
a wireless transmitter operable to communicate the digitized detected attenuated light signals to the monitor via a wireless communication link;
and wherein said system further includes:
a wireless receiver unit configured to connect to an input of the monitor to adapt the monitor to receive the digitized detected attenuated light signals via the wireless communication link.

33. The system of claim 22 wherein said sensor includes:
a cable configured to communicate the digitized detected attenuated light signals to the monitor.

34. The system of claim 33 further comprising:
an adaptor unit connectable with an input of the monitor and with the cable of the sensor, said adaptor unit being operable to convert the digitized detected attenuated light signals receivable from the cable of the sensor to an analog signal transmittable from the adaptor unit to the input of the monitor.

35. A method for use in obtaining information relating to a physiological condition of a patient from light signals attenuated by a tissue site of the patient, said method comprising:
operating a sensor located at a patient tissue site to direct at least two light signals into the patient tissue site, detect the light signals after the light signals are attenuated by the patient tissue site, and generate a digital signal corresponding to the attenuated light signals;
communicating the digital signal to a monitor separate from the sensor; and
processing the digital signal at the monitor to obtain information relating to the patient physiological condition.

36. The method of claim 35 wherein the at least two light signals are centered at Red and Infrared wavelengths, respectively.

37. The method of claim 35 wherein the patient physiological condition comprises at least one of a blood oxygen saturation level and a pulse rate of the patient.

38. The method of claim 35 wherein said step of communicating is performed using a wired communication link between the sensor and the monitor.

39. The method of claim 38 further comprising:
adapting the monitor to receive the digital signal via the wired communication link.

40. The method of claim 35 wherein said step of communicating is performed using a wireless communication link between the sensor and the monitor.

41. The method of claim 40 further comprising:
adapting the monitor to receive the digital signal via the wireless communication link.
